(19) United States
(12) Patent Application Publication
(10) Pub. No.: US 2003/0171331 A1
(43) Pub. Date: Sep. 11, 2003

(54) Oxazolidinone cotherapy

(76) Inventors: Joseph Patrick Martin JR., Richland, MI (US); Michael J. Dupuis, Mattawan, MI (US); John T. Herberg, Portage, MI (US)

Correspondence Address: PHARMACIA & UPJOHN, 301 HENRIETTA ST, 0228-32-LAW, KALAMAZOO, MI, 49007, US

(21) Appl. No.: 10/348,413

(22) Filed: Jan. 21, 2003

Related U.S. Application Data
(60) Provisional application No. 60/351,628, filed on Jan. 25, 2002.

(51) Int. Cl. A61K 31/714; A61K 31/541; A61K 31/5377; A61K 31/496; A61K 31/454; A61K 31/421; A61K 31/535; A61K 31/525

(52) U.S. Cl. 514/52; 514/251; 514/276; 514/376; 514/235.5; 514/254.02; 514/326; 514/227.5

(57) ABSTRACT

The present invention describes a novel cotherapy of oxazolidinones and at least one vitamin selected from vitamin B2, vitamin B6, vitamin B12 and folic acid.

### CROSS-REFERENCE TO RELATED APPLICATION

This application claims the benefit of the following provisional application(s): No. 60/351,628, filed Jan. 25, 2002, under 35 USC 119(e)(i).

### FIELD OF THE INVENTION

The present invention relates to a novel cotherapy which involves coadministration of oxazolidinone and at least one vitamin selected from the group consisting of vitamin B2, vitamin B6, vitamin B12 and folic acid. Furthermore, the invention refers to a respective pharmaceutical composition and a respective medical kit.

### BACKGROUND

Oxazolidinones are a well-known class of drugs which have been employed in a variety of applications. They are especially useful as antimicrobials with potent activity against a number of human and veterinary pathogens, including Gram-positive aerobic bacteria such as multiply-resistant staphylococci and streptococci, anaerobic organisms such as bacteroides and clostridia species, and acid-fast organisms such as Mycobacterium tuberculosis and Mycobacterium avium. More recently oxazolidinones demonstrating a useful level of activity against aerobic Gram-negative organisms such as Haemophilus influenza and Moraxella catarrhalis have also been described. For example, antibacterial oxazolidinones and methods for their preparation are described in the U.S. Pat. Nos. 5,225,565; 5,182,403; 5,164,510; 5,247,090; 5,231,188; 5,565,571; 5,668,286; 5,547,950; 5,952,324; 5,968,962; 5,688,792; 6,069,160; 6,239,152; 5,792,765; 4,705,799; 5,043,443; 5,652,238; 5,827,857; 5,529,998; 5,684,023; 5,627,181; 5,698,574; 6,166,056; 6,051,716; 6,255,304; 6,043,266; 6,313,307; and 5,523,403 as well as in the PCT Applications WO94/0110, WO95/07271, WO95/25106, WO96/13502, WO96/35691, WO97/09328, WO97/09328, WO97/10235, WO97/10223, WO97/19089, WO97/21708, WO97/30981, WO96/15130, WO96/23788, WO98/54161, WO99/29688, WO97/30995, WO97/09328, WO95/07271, WO00/21960, WO01/40236, WO99/64417, and WO01/81350.

Several classes of oxazolidinones are also known to have antidepressant activity. Some examples of these compounds are disclosed in the U.S. Pat. Nos. 5,714,502; 5,475,014, 4,250,318, 3,687,965; and 4,824,838 as well as U.S. Re. Pat. No. 29,607.

It has been observed that in a small number of patients oxazolidinones may cause some side effects. Potential side effects that might be associated with oxazolidinones are sideroblastic anemia, peripheral sensory neuropathy, optic neuropathy, seizures, thrombocytopenia, cheilosis, seborrheic dermatitis, hypo-regenerative anemia, megaloblastic anemia or normocytic anaemia. The patients in which the side effects were heretofore observed were generally on long-term treatment with oxazolidinones and were receiving treatment for a number of diseases and conditions apart from the condition (e.g. bacterial infection) for which the oxazolidinone was being administered. The patients who developed the side effects often also had a variety of other medical complications or predisposing conditions.

It is, therefore, an object of the present invention to prevent the occurrence of oxazolidinone-associated side effects in patients.

### SUMMARY OF THE INVENTION

In one embodiment the present invention refers to a method of treating a patient in need of oxazolidinone by administering an effective amount of oxazolidinone and an effective amount of at least one vitamin selected from the group consisting of vitamin B2, vitamin B6, vitamin B12 and folic acid.

A further embodiment of the invention is a method of treating or preventing an oxazolidinone-associated side effect by administering an effective amount at least one vitamin selected from the group consisting of vitamin B2, vitamin B6, vitamin B12 and folic acid to a patient in need thereof.

Other embodiments of the invention refer to methods of treating or preventing oxazolidinone-associated normocytic anemia or peripheral sensory neuropathy by administering an effective amount of vitamin B2 to a patient in need thereof.

Other embodiments of the invention refer to methods of treating or preventing oxazolidinone-associated sideroblastic anemia, peripheral sensory neuropathy, optic neuropathy, seizures, thrombocytopenia, cheilosis and seborrheic dermatitis by administering an effective amount of vitamin B6 to a patient in need thereof.

A method of treating or preventing oxazolidinone-associated hyporegenerative or megaloblastic anemia by administering an effective amount at least one vitamin selected from the group consisting of vitamin B12 and folic acid to a patient in need thereof is also described.

Yet another embodiment of the invention is a method of treating or preventing a bacterial infection by administering an effective amount of oxazolidinone and an effective amount of at least one vitamin selected from the group consisting of vitamin B2, vitamin B6, vitamin B12 and folic acid.

A method of treating or preventing a depressive disorder by administering an effective amount of oxazolidinone and an effective amount of at least one vitamin selected from the group consisting of vitamin B2, vitamin B6, vitamin B12 and folic acid is also disclosed.

Another embodiment of the invention refers to a pharmaceutical composition comprising (a) oxazolidinone and (b) at least one vitamin selected from the group consisting of vitamin B2, vitamin B6, vitamin B12 and folic acid.

A medical kit comprising (a) oxazolidinone and (b) at least one vitamin selected from the group consisting of vitamin B2, vitamin B6, vitamin B12 and folic acid is also referred to. In contrast to a pharmaceutical composition, in which all of the compounds are present in the same composition, at least two of the compounds are present in separate compositions in a medical kit.

### DESCRIPTION OF PREFERRED EMBODIMENTS

The invention resides in the surprising finding that oxazolidinone-associated side effects can be treated by administering at least one vitamin selected from the group consisting of vitamin B2, vitamin B6, vitamin B12 and folic acid. Consequently a patient in need of an oxazolidinone would not only receive the oxazolidinone but also at least one vitamin selected from the group consisting of vitamin B2, vitamin B6, vitamin B12 and folic acid.

“Oxazolidinone-associated side effect” means any disorder, abnormal condition or undesirable result caused by oxazolidinone administration, which can be treated by vitamin B2, vitamin B6, vitamin B12, folic acid or combinations thereof. Potential side effects which might be associated with oxazolidinones are normocytic anemia, sideroblastic anemia, peripheral sensory neuropathy, optic neuropathy, seizures, thrombocytopenia, hyporegenerative anemia, megaloblastic anemia, glossitis, cheilosis, stomatitis and seborrheic dermatitis.

In the context of the present invention, the term “oxazolidinone” is intended to mean any oxazolidinone which can be used in medical therapy. A variety of such oxazolidinones are known and their structures and methods of preparation are disclosed, for example, in U.S. Pat. Nos. 5,225,565; 5,182,403; 5,164,510; 5,247,090; 5,231,188; 5,565,571; 5,668,286; 5,547,950; 5,952,324; 5,968,962; 5,688,792; 6,069,160; 6,239,152; 5,792,765; 4,705,799; 5,043,443; 5,652,238; 5,827,857; 5,529,998; 5,684,023; 5,627,181; 5,698,574; 6,166,056; 6,051,716; 6,255,304; 6,043,266; 6,313,307; 5,523,403; 5,714,502; 5,475,014, 4,250,318, 3,687,965; and 4,824,838, the U.S. Re. Pat. No. 29,607 as well as in the PCT Applications WO94/01110, WO95/07271, WO95/25106, WO96/13502, WO96/35691, WO97/09328, WO97/09328, WO97/10235, WO97/10223, WO97/19089, WO97/21708, WO97/30981, WO96/15130, WO96/23788, WO98/54161, WO99/29688, WO97/30995, WO97/09328, WO95/07271, WO00/21960, WO01/40236, WO99/64417, and WO01/81350, which are incorporated herein by reference in their entirety. A number of oxazolidinones are commercially available as pharmaceuticals. Examples are linezolid (available from PHARMACIA Corp. as Zyvox), furazolidone (available from Roberts Pharmaceuticals as Furoxone) and toloxatone (available from Sanofi-Synthelabo as Humoryl).

A class of oxazolidinones which can be especially referred to in the context of the present invention are the oxazolidinones of the general formula I:

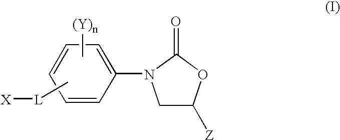

wherein

X is selected from the group consisting of a C₁₋₁₀ alkyl group (the alkyl group optionally being substituted with at least one substituent R⁴), a C₂₋₁₀ alkenyl group (the alkenyl group optionally being substituted with at least one substituent R⁴), a C₂₋₁₀ alkynyl group (the alkynyl group optionally being substituted with at least one substituent R⁴), a C₃₋₇ cycloalkyl group (the cycloalkyl group optionally being substituted with at least one substituent R⁵), a C₃₋₇ cycloalkenyl group (the cycloalkenyl group optionally being substituted with at least one substituent R⁵), a C₅₋₇ aryl group (the aryl group optionally being substituted with at least one substituent R⁵), and a saturated or unsaturated C₃₋₇ heterocyclic group containing at least one heteroatom selected from O, S, and N (the heterocyclic group optionally being substituted with at least one substituent R⁵);

L is an organic linking group selected from the group consisting of a covalent bond, —O—, —S—, —C(O)—, —C(O)O—, —OC(O)—, —NR¹—, —C(O)NR¹—, —NR¹C(O)—, a C₁₋₄ alkylene group, a C₂₋₄ alkenylene group and a C₂₋₄ alkynylene group (wherein one of the (CH₂) moieties in the alkylene group, alkenylene group or alkynylene group can optionally be replaced by —O—, —S—, —C(O)—, —C(O)O—, —OC(O)—, —NR¹—, —C(O)NR¹— or —NR¹C(O)—) (preferably L is a covalent bond, —O—, —S—, —(CH₂)ₜ—, —(CH₂)ₜ—O— or —(CH₂), —S— in which t is 1 or 3; in one preferred embodiment L is a covalent bond, —CH₂—CH₂— or —CH₂—O—; in another preferred embodiment L is a covalent bond);

Y is selected from the group consisting of halogen, —NR¹R², —CN, —NO₂, —OR¹, —SR¹, —S(O)R¹, —S(O)₂R¹, —OS(O)₂R¹, —S(O)₂NR¹R², —NR¹S(O)₂R¹, —C(O)OR¹, —OC(O)R¹, —COR¹, —CONR¹R², —NR¹COR², a C₁₋₆ alkyl group (the alkyl group optionally being substituted with at least one substituent R³), a C₁₋₆ ether group, a C₁₋₆ thioether group, a C₂₋₆ alkenyl group (the alkenyl group optionally being substituted with at least one substituent R³), and a C₂₋₆ alkynyl group (the alkynyl group optionally being substituted with at least one substituent R³) (preferably Y is selected from the group consisting of halogen, —NR¹R², —OR¹, —C(O)OR¹R², a C₁₋₆ alkyl group (the alkyl group optionally being substituted with at least one halogen or —OH), and a C₁₋₆ ether group; more preferably Y is halogen, a C₁₋₄ alkyl group (the alkyl group optionally being substituted with at least one halogen), and a C₁₋₄ alkoxy group; it is further preferred for Y to be —F, —Cl or a C₁₋₄ alkyl group optionally substituted with F or Cl; in one preferred embodiment Y is —F, —Cl, —CH₃ or —CF₃; in another preferred embodiment Y is —F);

n is in the range of 0 to 4 (preferably n is 0, 1 or 2; more preferably the groups Y, if present, are meta to the oxazolidinone ring; in a preferred embodiment n is 1);

Z is selected from the group consisting of —CH₂—O—R⁸, —CH₂—NH—C(O)—R⁹, —CH₂—S—R⁸ and —CH₂—NH—C(S)—R⁹ (in one preferred embodiment Z is —CH₂—O—R⁸ and in a further preferred embodiment Z is —CH₂—NH—C(O)—R⁹, in a further embodiment Z is —CH₂—S—R⁸ and in another embodiment Z is —CH₂—NH—C(S)—R⁹); and

R¹ and R² are independently hydrogen or a C₁₋₆ alkyl group (the alkyl group optionally being substituted with at least one halogen, —OH, C₁₋₆ alkoxy group, —NH₂, C₁₋₆ alkylamino group or C₁₋₆ dialkylamino group) (preferably R¹ and R² are hydrogen or a C₁₋₆ alkyl group (the alkyl group optionally being substituted with at least one halogen or —OH); more preferably R¹ and R² are hydrogen, a C₁₋₄ alkyl group or a C₁₋₄ alkyl group substituted with one or two —OH);

R³ is selected from the group consisting of halogen, —OH, —NH₂, a C₁₋₆ alkylamino group, a C₁₋₆ dialkylamino group, a C₁₋₆ alkoxy group, a C₁₋₆ acyloxy group and a benzyloxy group (R³ is preferably selected from the group consisting of halogen, —OH, —NH₂, a C₁₋₆ alkyl group, a C₁₋₆ alkylamino group, a C₁₋₆ dialkylamino group, and a C₁₋₆ alkoxy group; more preferably R³ is selected from the group consisting of halogen, —OH or a C₁₋₃ alkoxy group; even more preferably R³ is halogen);

R^{3a} is selected from the group consisting of halogen, —OH, —NH₂, a C₁₋₆ alkylamino group, a C₁₋₆ dialkylamino group, a C₁₋₆ alkoxy group, a C₁₋₆ acyloxy group, a benzyloxy group, a C₃₋₆ cycloalkyl group (the cycloalkyl group optionally being substituted with at least one halogen, —OH or —NH₂), a C₃₋₆ cycloalkenyl group (the cycloalkenyl group optionally being substituted with at least one halogen, —OH or —NH₂), a C₅₋₇ aryl group (the aryl group optionally being substituted with at least one halogen, —OH or —NH₂), and a saturated or unsaturated C₃₋₇ heterocyclic group containing at least one heteroatom selected from O, S, and N (the heterocyclic group optionally being substituted with at least one halogen, —OH or —NH₂); (R^{3a} is preferably selected from the group consisting of halogen, —OH, —NH₂, a C₁₋₆ alkyl group, a C₁₋₆ alkylamino group, a C₁₋₆ dialkylamino group, a C₁₋₆ alkoxy group, a C₅₋₇ aryl group (the aryl group optionally being substituted with at least one halogen, —OH or —NH₂), and a saturated or unsaturated C₃₋₇ heterocyclic group containing at least one heteroatom selected from O, S, and N (the heterocyclic group optionally being substituted with at least one halogen, —OH or —NH₂); more preferably R^{3a} is selected from the group consisting of halogen, —OH or a C₁₋₃ alkoxy group; even more preferably R^{3a} is halogen);

R^{3b} is selected from the group consisting of halogen, —OH, —NH₂, a C₁₋₆ alkylamino group, a C₁₋₆ dialkylamino group, a C₁₋₆ alkoxy group, a C₁₋₆ acyloxy group, a benzyloxy group a C₁₋₆ alkyl group (the alkyl group optionally being substituted with at least one halogen, —OH or —NH₂), a C₁₋₆ ether group, a C₁₋₆ thioether group, a C₂₋₆ alkenyl group (the alkenyl group optionally being substituted with at least one halogen, —OH or —NH₂), and a C₂₋₆ alkynyl group (the alkynyl group optionally being substituted with at least one halogen, —OH or —NH₂); (R^{3b} is preferably selected from the group consisting of halogen, —OH, —NH₂, a C₁₋₆ alkyl group, a C₁₋₆ alkylamino group, a C₁₋₆ dialkylamino group, and a C₁₋₆ alkoxy group; more preferably R^{3b} is selected from the group consisting of halogen, —OH or a C₁₋₃ alkoxy group; even more preferably R^{3b} is halogen);

R⁴ is selected from the group consisting of halogen, —NR¹R², —CN, —NO₂, —OR¹, —SR¹, —S(O)R¹, —S(O)₂R¹, —OS(O)₂R¹, —S(O)₂NR¹R², —NR¹S(O)₂R², —C(O)OR¹, —OC(O)R¹, —COR¹, —CONR¹R², —NR¹COR², a p-toluenesulfonyl group, a C₃₋₆ cycloalkyl group (the cycloalkyl group optionally being substituted with at least one substituent R³), a C₃₋₆ cycloalkenyl group (the cycloalkenyl group optionally being substituted with at least one substituent R³), a C₅₋₇ aryl group (the aryl group optionally being substituted with at least one substituent R³), and a saturated or unsaturated C₃₋₇ heterocyclic group containing at least one heteroatom selected from O, S, and N (the heterocyclic group optionally being substituted with at least one substituent R³) (preferably R⁴ is selected from the group consisting of halogen, —NR¹R², —CN, —NO₂, —OR¹, —SR¹, —C(O)OR¹, —OC(O)R¹, —COR¹, —CONR¹R², a C₃₋₆ cycloalkyl group (the cycloalkyl group optionally being substituted with at least one substituent R³), a C₃₋₆ cycloalkenyl group (the cycloalkenyl group optionally being substituted with at least one substituent R³), a C₅₋₇ aryl group (the aryl group optionally being substituted with at least one substituent R³), and a saturated or unsaturated C₃₋₇ heterocyclic group containing at least one heteroatom selected from O, S, and N (the heterocyclic group optionally being substituted with at least one substituent R³); more preferably R⁴ is halogen or —OH);

R⁵ is selected from the group consisting of halogen, —NR¹R², ═O, ═S, ═N—R¹, —CN, —NO₂, —OR¹, —SR¹, —S(O)R¹, —S(O)₂R¹, —OS(O)₂R¹, —S(O)₂NR¹R², —NR¹S(O)₂R², —C(O)OR¹, —OC(O)R¹, —COR¹, —CONR¹R², —NR¹COR², a p-toluenesulfonyl group, a C₁₋₆ alkyl group (the alkyl group optionally being substituted with at least one substituent R³), a C₁₋₆ ether group, a C₁₋₆ thioether group, a C₂₋₆ alkenyl group (the alkenyl group optionally being substituted with at least one substituent R³), a C₂₋₆ alkynyl group (the alkynyl group optionally being substituted with at least one substituent R³), a C₃₋₆ cycloalkyl group (the cycloalkyl group optionally being substituted with at least one substituent R³), a C₃₋₆ cycloalkenyl group (the cycloalkenyl group optionally being substituted with at least one substituent R^{3b}), a C₅₋₇ aryl group (the aryl group optionally being substituted with at least one substituent R³), and a saturated or unsaturated C₃₋₇ heterocyclic group containing at least one heteroatom selected from O, S, and N (the heterocyclic group optionally being substituted with at least one substituent R³) (preferably R⁵ is selected from the group consisting of halogen, —NR¹R², ═O, ═S, ═N—R¹, —CN, —NO₂, —C(O)OR¹, —C(O)R¹, and a C₁₋₆ alkyl group (the alkyl group optionally being substituted with at least one substituent R³));

R⁸ is selected from the group consisting of hydrogen, a C₁₋₆ alkyl group (the alkyl group optionally being substituted with at least one substituent R^{3a}), a C₁₋₆ ether group, a C₁₋₆ thioether group, a C₂₋₆ alkenyl group (the alkenyl group optionally being substituted with at least one substituent R^{3a}), a C₂₋₆ alkynyl group (the alkynyl group optionally being substituted with at least one substituent R^{3a}), a C₃₋₆ cycloalkyl group (the cycloalkyl group optionally being substituted with at least one substituent R^{3b}), a C₃₋₆ cycloalkenyl group (the cycloalkenyl group optionally being substituted with at least one substituent R^{3b}), a C₅₋₇ aryl group (the aryl group optionally being substituted with at least one substituent R^{3b}), and a saturated or unsaturated C₃₋₇ heterocyclic group containing at least one heteroatom selected from O, S, and N (the heterocyclic group optionally being substituted with at least one substituent R^{3b}) (preferably R⁸ is selected from the group consisting of hydrogen, a C₁₋₆ alkyl group (the alkyl group optionally being substituted at least one of the following: halogen, —OH, a C₁₋₆ alkoxy group, a C₁₋₆ acyloxy group, —NH₂, a C₁₋₆ alkylamino group, a C₁₋₆ dialkylamino group, and a benzyloxy group), a C₂₋₆ alkenyl group, a C₃₋₆ cycloalkyl group, a C₃₋₆ cycloalkenyl group, a C₅₋₇ aryl group, and a saturated or unsaturated C₅₋₇ heterocyclic group containing at least one heteroatom selected from O, S, and N; more preferably R⁸ is selected from the group consisting of hydrogen, a C₁₋₆ alkyl group (the alkyl group optionally being substituted with at least one hydroxy), a C₅₋₆ cycloalkyl group, a C₅₋₆ cycloalkenyl group, a C₅₋₇ aryl group, and a saturated or unsaturated C₅₋₇ heterocyclic group containing at least one heteroatom selected from O, S and N); and

R⁹ is selected from the group consisting of hydrogen, —NH₂, a C₁₋₆ alkylamino group, a C₁₋₆ dialkylamino group, a C₁₋₆ alkoxy group, a C₁₋₆ alkyl group (the alkyl group optionally being substituted with at least one substituent R^{3a}), a C₁₋₆ ether group, a C₁₋₆ thioether group, a C₂₋₆ alkenyl group (the alkenyl group optionally being substituted with at least one substituent R^{3a}), a C₂₋₆ alkynyl group (the alkynyl group optionally being substituted with at least one substituent R^{3a}), a C₃₋₆ cycloalkyl group (the cycloalkyl group optionally being substituted with at least one substituent R^{3b}), a C₃₋₆ cycloalkenyl group (the cycloalkenyl group optionally being substituted with at least one substituent R^{3b}), a C₅₋₇ aryl group (the aryl group optionally being substituted with at least one substituent R^{3b}), and a saturated or unsaturated C₃₋₇ heterocyclic group containing at least one heteroatom selected from O, S, and N (the heterocyclic group optionally being substituted with at least one substituent R^{3b}) (preferably R⁹ is selected from the group consisting of a C₁₋₆ alkyl group (the alkyl group optionally being substituted by at least one of the following: halogen, —OH, a C₁₋₆ alkoxy group, a C₁₋₆ acyloxy group, a benzyloxy group, a C₅₋₇ aryl group (the aryl group optionally being substituted with at least one halogen, —OH or —NH₂), and a saturated or unsaturated C₃₋₇ heterocyclic group containing at least one heteroatom selected from O, S, and N (the heterocyclic group optionally being substituted with at least one halogen, —OH or —NH₂)), a C₂₋₆ alkenyl group (the alkenyl group optionally being substituted with at least one of the following: halogen, —OH, a C₁₋₆ alkoxy group, a C₁₋₆ acyloxy group, a benzyloxy group, a C₅₋₇ aryl group (the aryl group optionally being substituted with at least one halogen, —OH or —NH₂), and a saturated or unsaturated C₃₋₇ heterocyclic group containing at least one heteroatom selected from O, S, and N (the heterocyclic group optionally being substituted with at least one halogen, —OH or —NH₂)), —NH₂, a C₁₋₆ alkylamino group, a C₁₋₆ dialkylamino group, a C₁₋₆ alkoxy group, a C₃₋₆ cycloalkyl group, a C₃₋₆ cycloalkenyl group, a C₅₋₇ aryl group, and a saturated or unsaturated C₅₋₇ heterocyclic group containing at least one heteroatom selected from O, S and N; more preferably R⁹ is a C₁₋₄ alkyl group (the alkyl group optionally being substituted with at least one —F, —Cl, —OH).

In the context of the present invention the expression “at least one substituent” means that one substituent or any other number of substituents up to the maximum chemically possible number of substituents can be present on the group, which is substituted. Unless otherwise mentioned the number of substituents, if present, is preferably 1, 2 or 3, more preferably the number of substituents is 1 or 2. Generally, however, unless otherwise mentioned, the groups which are defined as being optionally substituted are preferably unsubstituted.

In the present invention a preferred C₅₋₇ aryl group is phenyl.

A saturated or unsaturated heterocyclic group containing at least one heteroatom selected from O, S, and N can be any heterocyclic group, which is saturated, unsaturated or aromatic. It can contain any chemically possible number of heteroatoms, but preferably includes 1, 2 or 3 (more preferably 1 or 2) heteroatoms. The heteroatoms can be solely included in the ring (e.g. in the form of —O—, —S—, —NR—, —N═, >N—N<, —N═N—, >N—N—N< or —N═N—N< groups) or can be present in heteroatom-containing groups such as, but not restricted to, —S(O)—, —S(O)₂—, —OS(O)₂—, —S(O)₂NR—, —NRS(O)₂R—, —C(O)O—, —C(O)OC(O)—, —CONR— or —RNCONR—. In addition to the above, the heterocyclic group can have heteroatoms, e.g. in the form of —C(═NR)—, —C(═O)— and —C(═S)—. Examples of heterocyclic groups include, but are not limited to, pyridyl, thienyl, furyl, pyrazolyl, pyrimidyl, 2-pyridyl, 3-pyridyl, 4-pyridyl, 2-pyrimidinyl, 4-pyrimidinyl, 5-pyrimidinyl, 3-pyridazinyl, 4-pyridazinyl, 3-pyrazinyl, 4-oxo-2-imidazolyl, 2-imidazolyl, 4-imidazolyl, 3-isoxazolyl, 4-isoxazolyl, 5-isoxazolyl, 3-pyrazolyl, 4-pyrazolyl, 5-pyrazolyl, 2-oxazolyl, 4-oxazolyl, 4-oxo-2-oxazolyl, 5-oxazolyl, 1,2,3-oxathiazole, 1,2,3-oxadiazole, 1,2,4-oxadiazole, 1,2,5-oxadiazole, 1,3,4-oxadiazole, 2-thiazolyl, 4-thiazolyl, 5-thiazolyl, 3-isothiazole, 4-isothiazole, 5-isothiazole, 2-furanyl, 3-furanyl, 2-thienyl, 3-thienyl, 2-pyrrolyl, 3-pyrrolyl, 3-isopyrrolyl, 4-isopyrrolyl, 5-isopyrrolyl, 1,2,3,-oxathiazole-1-oxide, 1,2,4-oxadiazol-3-yl, 1,2,4-oxadiazol-5-yl, 5-oxo-1,2,4-oxadiazol-3-yl, 1,2,4-thiadiazol-3-yl, 1,2,4-thiadiazol-5-yl, 3-oxo-1,2,4-thiadiazol-5-yl, 1,3,4-thiadiazol-5-yl, 2-oxo-1,3,4-thiadiazol-5-yl, 1,2,4-triazol-3-yl, 1,2,4-triazol-5-yl, 1,2,3,4-tetrazol-5-yl, 5-oxazolyl, 3-isothiazolyl, 4-isothiazolyl, 5-isothiazolyl, 1,3,4,-oxadiazole, 4-oxo-2-thiazolinyl, 5-methyl-1,3,4-thiadiazol-2-yl, thiazoledione, 1,2,3,4-thiatriazole, 1,2,4-dithiazolone, azacyclopentyl, diazacyclopentyl, azacyclohexyl, diazacyclohexyl, azacyclopentenyl, diazacyclopentenyl, azacyclohexenyl, diazacyclohexenyl, oxacyclopentyl, dioxacyclopentyl, oxacyclohexyl, dioxacyclohexyl, oxacyclopentenyl, dioxacyclopentenyl, oxacyclohexenyl, dioxacyclohexenyl, azacyclopentanonyl, azacyclohexanonyl, azacyclopentenonyl, azacyclohexenonyl, oxacyclopentanonyl, oxacyclohexanonyl, oxacyclopentenonyl, oxacyclohexenonyl, pyridonyl, morpholinyl, diazinyl, triazinyl, quinolinyl, quinoxalinyl, and pyrrolidinyl.

For the purposes of the present invention, the term “halogen” means any of —F, —Cl, —Br or —I. Preferred halogens are —F and —Cl, most preferred is —F.

In one preferred embodiment of general formula I X can be selected from the group consisting of a C₁₋₁₀ alkyl group (the alkyl group optionally being substituted with at least one substituent R⁴), a C₂₋₁₀ alkenyl group (the alkenyl group optionally being substituted with at least one substituent R⁴), a C₂₋₁₀ alkynyl group (the alkynyl group optionally being substituted with at least one substituent R⁴). More preferably X can be a C₁₋₆ alkyl group (the alkyl group optionally being substituted with at least one R⁰⁷), a C₂₋₆ alkenyl group (the alkenyl group optionally being substituted with at least one substituent R⁰⁷) or a C₂₋₆ alkynyl group (the alkynyl group optionally being substituted with at least one substituent R⁰⁷). In these two embodiments L can be a covalent bond.

In a further preferred embodiment X can be

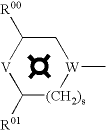

wherein

V is selected from the group consisting of —O—, —S—, —S(O)—, —S(O)₂—, —C(O)—, —C(S)—, —C(NR⁰³)—, —C(H)(NR¹³₂)—, —S(NR⁰³)—, —S(O)(NR⁰³)—, —C(H)(R⁰⁴)—, ═C(R⁰⁴)—, —N(R⁰⁴)— and —N═ (preferably V is selected from the group consisting of —O—, —S—, —S(O)—, —S(O)₂—, —C(H)(R⁰⁴)—, ═C(R⁰⁴)—, —NR⁰⁴— or —N═);

W is C, CH or N;

[symbol] means that the cyclic group containing V and W can be saturated or unsaturated;

R⁰⁰ is selected from the group consisting of —H, a C₁₋₄ alkyl group, —CN, and —C(O)OR⁰⁶ (preferably R⁰⁰ is —H);

R⁰¹ is —H or a C₁₋₄ alkyl group (preferably R⁰¹ is —H);

R⁰³ is hydrogen or a C₁₋₆ alkyl group (the alkyl group optionally being substituted with at least one halogen, —OH, C₁₋₆ alkoxy group, —NH₂, C₁₋₆ alkylamino group or C₁₋₆ dialkylamino group) (preferably R⁰³ is hydrogen or a C₁₋₄ alkyl group; more preferably R⁰³ is hydrogen);

R⁰⁴ is selected from the group consisting of hydrogen, a C₁₋₆ alkyl group (the alkyl group optionally being substituted with at least one halogen, —OH, C₁₋₆ alkoxy group, —NH₂, C₁₋₆ alkylamino group or C₁₋₆ dialkylamino group), and —C(O)R⁰⁵ (preferably R⁰⁴ is hydrogen, a C₁₋₄ alkyl group or —C(O)R⁰⁵);

R⁰⁵ is a C₁₋₆ alkyl group (the alkyl group optionally being substituted with at least one halogen, —OH, C₁₋₆ alkoxy group, —NH₂, C₁₋₆ alkylamino group or C₁₋₆ dialkylamino group) (preferably R⁰⁵ is a C₁₋₄ alkyl group optionally being substituted with one or two —OH);

R⁰⁶ is hydrogen or a C₁₋₄ alkyl group;

R⁰⁷ is selected from the group consisting of halogen, —CN, —NO₂, —NH₂, a C₁₋₆ alkylamino group, a C₁₋₆ dialkylamino group, —OR⁰⁸, —C(O)OR⁰⁸, —OC(O)R⁰⁸, —COR⁰⁸, —CONR⁰⁸₂, a C₅₋₇ aryl group (the aryl group optionally being substituted with at least one substituent R⁰⁹), and a saturated or unsaturated C₅₋₇ heterocyclic group containing at least one heteroatom selected from O, S, and N (R⁰⁷ is preferably selected from the group consisting of halogen, —CN, —NO₂, —NH₂, a C₁₋₆ alkylamino group, a C₁₋₆ dialkylamino group, —OR⁰⁸, —C(O)OR⁰⁸, —OC(O)R⁰⁸, —COR⁰⁸, —CONR⁰⁸₂; more preferably R⁰⁷ is halogen);

R⁰⁸ is hydrogen or a C₁₋₆ alkyl group;

R⁰⁹ is selected from the group consisting of halogen, —OH, —NH₂, a C₁₋₆ alkyl group, a C₁₋₆ alkylamino group, a C₁₋₆ dialkylamino group, a C₁₋₆ alkoxy group, a C₁₋₆ acyloxy group and a benzyloxy group (R⁰⁹ is preferably selected from the group consisting of halogen, —OH, —NH₂, a C₁₋₆ alkyl group, a C₁₋₆ alkylamino group, a C₁₋₆ dialkylamino group, and a C₁₋₆ alkoxy group; more preferably R⁰⁹ is halogen);

s is 0, 1 or 2 (preferably s is 0 or 1, more preferably s is 1); and

t is 1 or 2.

In one embodiment the oxazolidinone can be of the general formula (IIa) or (IIb):

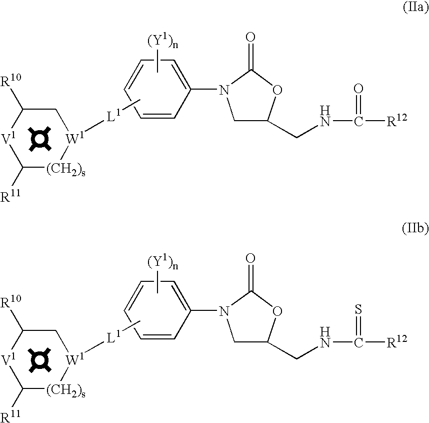

wherein:

L¹ is selected from the group consisting of a covalent bond, —(CH₂)ₜ— and —(CH₂)ₜ—O— (in one preferred embodiment L¹ is a covalent bond, —CH₂—CH₂— or —CH₂—O—; in another preferred embodiment L¹ is a covalent bond);

V¹ is selected from the group consisting of —O—, —S—, —S(O)—, —S(O)₂—, —C(O)—, —C(S)—, —C(NR¹³)—, —C(H)(NR¹³₂)—, —S(NR¹³)—, —S(O)(NR¹³)—, —C(H)(R¹⁴)—, —C(R¹⁴)═, —N(R¹⁴)— and —N═ (preferably V¹ is selected from the group consisting of —O—, —S—, —S(O)—, —S(O)₂—, —C(H)(R¹⁴)—, —C(R¹⁴)═, —N(R¹⁴)— and —N═; more preferably V¹ is selected from the group consisting of —O—, —S(O)₂—, —CHR¹⁴—, —CR¹⁴═, —NR¹⁴— and —N═);

W¹ is C, CH or N (preferably W¹ is N);

[symbol] means that the cyclic group containing V¹ and W¹ can be saturated or unsaturated (preferably the cyclic group containing V¹ and W¹ is saturated);

Y¹ is selected from the group consisting of halogen, a C₁₋₄ alkyl group (the alkyl group optionally being substituted with at least one halogen), and a C₁₋₄ alkoxy group (preferably Y¹ is —F, —Cl or a C₁₋₂ alkyl group (the alkyl group optionally being substituted with F and Cl); in a preferred embodiment Y¹ is —F, —Cl, —CH₃ or —CF₃; in another preferred embodiment Y¹ is —F);

R¹⁰ is selected from the group consisting of —H, a C₁₋₄ alkyl group, —CN, and —C(O)OR¹⁶ (preferably R¹⁰ is —H);

R¹¹ is —H or a C₁₋₄ alkyl group (preferably R¹¹ is —H);

R¹² is selected from the group consisting of hydrogen, a C₁₋₆ alkyl group (the alkyl group optionally being substituted by at least one of the following: halogen, —OH, a C₁₋₆ alkoxy group, a C₁₋₆ acyloxy group, a benzyloxy group, a C₅₋₇ aryl group (the aryl group optionally being substituted with at least one halogen, —OH or —NH₂), and a saturated or unsaturated C₃₋₇ heterocyclic group containing at least one heteroatom selected from O, S, and N (the heterocyclic group optionally being substituted with at least one halogen, —OH or —NH₂)), a C₂₋₆ alkenyl group (the alkenyl group optionally being substituted with at least one of the following: halogen, —OH, a C₁₋₆ alkoxy group, a C₁₋₆ acyloxy group, a benzyloxy group, a C₅₋₇ aryl group (the aryl group optionally being substituted with at least one halogen, —OH or —NH₂), and a saturated or unsaturated C₃₋₇ heterocyclic group containing at least one heteroatom selected from O, S, and N (the heterocyclic group optionally being substituted with at least one halogen, —OH or —NH₂)), —NH₂, a C₁₋₆ alkylamino group, a C₁₋₆ dialkylamino group, a C₁₋₆ alkoxy group, a C₃₋₆ cycloalkyl group, a C₃₋₆ cycloalkenyl group, a C₅₋₇ aryl group, and a saturated or unsaturated C₅₋₇ heterocyclic group containing at least one heteroatom selected from O, S and N (preferably R¹² is a C₁₋₄ alkyl group (the alkyl group optionally being substituted with at least one —F, —Cl or —OH);

R¹³ is hydrogen or a C₁₋₆ alkyl group (the alkyl group optionally being substituted with at least one halogen, —OH, C₁₋₆ alkoxy group, —NH₂, C₁₋₆ alkylamino group or C₁₋₆ dialkylamino group) (preferably R¹³ is hydrogen or a C₁₋₄ alkyl group, more preferably R¹³ is hydrogen);

R¹⁴ is selected from the group consisting of hydrogen, a C₁₋₆ alkyl group (the alkyl group optionally being substituted with at least one halogen, —OH, C₁₋₆ alkoxy group, —NH₂, C₁₋₆ alkylamino group or C₁₋₆ dialkylamino group), and —C(O)R¹⁵ (preferably R¹⁴ is hydrogen, a C₁₋₄ alkyl group or —C(O)R¹⁵);

R¹⁵ is a C₁₋₆ alkyl group (the alkyl group optionally being substituted with at least one halogen, —OH, C₁₋₆ alkoxy group, —NH₂, C₁₋₆ alkylamino group or C₁₋₆ dialkylamino group) (preferably R¹⁵ is a C₁₋₄ alkyl group optionally being substituted with one or two —OH);

R¹⁶ is hydrogen or a C₁₋₄ alkyl group;

n is in the range of 0 to 4 (preferably n is 0, 1 or 2; more preferably the groups Y¹, if present, are meta to the oxazolidinone ring; in a preferred embodiment n is 1);

s is 0, 1 or 2 (preferably s is 0 or 1, more preferably s is 1); and

t is 1 or 2.

Examples of individual preferred compounds are (S)-N-[[3-[3-fluoro-4-(4-thiomorpholinyl)phenyl]-2-oxo-5-oxazolidinyl]methyl]acetamide; (S)-N-[[3-[3-fluoro-4-(1,1-dioxothiomorpholin-4-yl)phenyl]-2-oxo-5-oxazolidinyl]methyl]acetamide; (S)-N-[[3-[3-fluoro-4-(1-oxothiomorpholin-4-yl)phenyl]-2-oxo-5-oxazolidinyl]methyl]acetamide; (S)-N-[[3-[3,5-difluoro-4-morpholinyl]phenyl]-2-oxo-5-oxazolidinyl]methyl]acetamide; (S)-N-[[3-[3-fluoro-4-morpholinyl]phenyl]-2-oxo-5-oxazolidinyl]methyl]acetamide; (S)-N-[[3-[3-fluoro-4-[1-[(p-toluenesulfonyl)imino]thiomorpholin-4-yl]phenyl]-2-oxo-5-oxazolidinyl]methyl]acetamide; (S)-N-[[3-[3-fluoro-4-morpholinyl]phenyl]-2-oxo-5-oxazolidinyl]methyl]hydroxyacetamide; (S)-N-[[3-[3-fluoro-4-morpholinyl]phenyl]-2-oxo-5-oxazolidinyl]methyl]formamide; (S)-N-[[3-[3-fluoro-4-morpholinyl]phenyl]-2-oxo-5-oxazolidinyl]methyl]methylcarbamate; (S)-N-[[3-[3-fluoro-4-morpholinyl]phenyl]-2-oxo-5-oxazolidinyl]methyl]dichloroacetamide; (S)-N-[[3-[3-fluoro-4-(3-thiazolidinyl)phenyl]-2-oxo-5-oxazolidinyl]methyl]acetamide; (S)-N-[[3-[3-fluoro-4-(1,1-dioxothiazolidin-3-yl)phenyl]-2-oxo-5-oxazolidinyl]methyl]acetamide; (S)-N-[[3-[3-fluoro-4-(1-oxothiazolidin-3-yl)phenyl]-2-oxo-5-oxazolidinyl]methyl]acetamide; (S)-N-[[3-[3-fluoro-4-(3-oxazolidinyl)]phenyl]-2-oxo-5-oxazolidinyl]methyl]acetamide; (S)-N-[[3-[3-fluoro-4-(hexahydrothiazepin-4-yl)phenyl]-2-oxo-5-oxazolidinyl]methyl]acetamide; (S)-N-[[3-[3-fluoro-4-(1,1-dioxohexahydrothiazepin-4-yl)phenyl]-2-oxo-5-oxazolidinyl]methyl]acetamide; (S)-N-[[3-[3-fluoro-4-(1-oxohexahydrothiazepin-4-yl)phenyl]-2-oxo-5-oxazolidinyl]methyl]acetamide; 2,2-difluoro-N-({(5S)-3-[3-fluoro-4-(4-glycoloyl piperazin-1-yl)phenyl]-2-oxo-1,3-oxazolidin-5-yl}methyl)ethanethioamide; and (S)-N-[[3-[3-fluoro-4-(hexahydrooxazepin-4-yl)]phenyl]-2-oxo-5-oxazolidinyl]methyl]acetamide. In particular the oxazolidinone can be

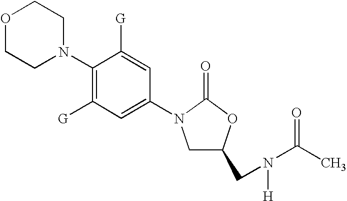

wherein G are independently selected from the group consisting of —H, —F, —Cl, —CH₃ and —CF₃.

In particular the oxazolidinone can be

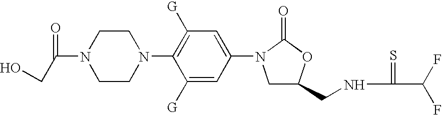

wherein G are independently selected from the group consisting of —H and —F.

In a further embodiment the oxazolidinone can have the general formula (IIIa) or

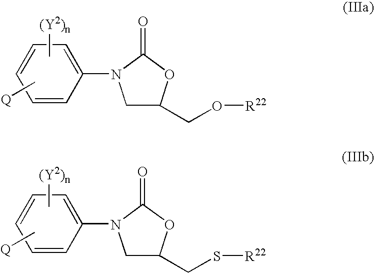

wherein:

Q can be selected from the group consisting of a C₁₋₆ alkyl group (the alkyl group optionally being substituted with at least one substituent R²⁷), a C₂₋₆ alkenyl group (the alkenyl group optionally being substituted with at least one substituent R²⁷), a C₂₋₆ alkynyl group (the alkynyl group optionally being substituted with at least one substituent R²⁷) or Q can be

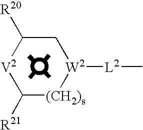

wherein

L² is selected from the group consisting of a covalent bond, —(CH₂)ₜ— and —(CH₂)ₜ—O— (in one preferred embodiment L² is a covalent bond, —CH₂—CH₂— or —CH₂—O—; in another preferred embodiment L² is a covalent bond);

V² is selected from the group consisting of —O—, —S—, —S(O)—, —S(O)₂—, —C(O)—, —C(S)—, —C(NR²³)—, —C(H)(NR²³₂)—, —S(NR²³)—, —S(O)(NR²³)—, —C(H)(R²⁴)—, —C(R²⁴)═, —N(R²⁴)— and —N═ (preferably V² —O—, —S—, —S(O)—, —S(O)₂—, —CHR²⁴—, —CR²⁴═, —NR²⁴— and —N═; more preferably V² is selected from the group consisting of —O—, —C(H)(R²⁴)—, —C(R²⁴)═, —N(R²⁴)— and —N═);

W is C, CH or N (preferably W² is C or CH; most preferably W² is C);

[symbol] means that the cyclic group containing V² and W² can be saturated or unsaturated (preferably the cyclic group is unsaturated and contains at least one double bond; most preferably the cyclic group is a cyclohexenyl group containing one double bond or is a phenyl ring);

Y² is selected from the group consisting of halogen, a C₁₋₄ alkyl group (the alkyl group optionally being substituted with at least one halogen), and a C₁₋₄ alkoxy group (preferably Y² is —F, —Cl or a C₁₋₂ alkyl group (the alkyl group optionally being substituted with F and Cl); in a preferred embodiment Y² is —F, —Cl, —CH₃ or —CF₃; in another preferred embodiment Y² is —F);

R²⁰ is selected from the group consisting of —H, a C₁₋₄ alkyl group, —CN, and —C(O)OR²⁶ (preferably R²⁰ is —H);

R²¹ is —H or a C₁₋₄ alkyl group (preferably R²¹ is —H);

R²² is selected from the group consisting of hydrogen, a C₁₋₆ alkyl group (the alkyl group optionally being substituted at least one of the following: halogen, —OH, a C₁₋₆ alkoxy group, a C₁₋₆ acyloxy group, —NH₂, a C₁₋₆ alkylamino group, a C₁₋₆ dialkylamino group, a benzyloxy group), a C₃₋₆ cycloalkyl group, a C₃₋₆ cycloalkenyl group, a C₅₋₇ aryl group, and a saturated or unsaturated C₅₋₇ heterocyclic group containing at least one heteroatom selected from O, S and N;

R²³ is hydrogen or a C₁₋₆ alkyl group (the alkyl group optionally being substituted with at least one halogen, —OH, C₁₋₆ alkoxy group, —NH₂, C₁₋₆ alkylamino group or C₁₋₆ dialkylamino group) (preferably R²³ is hydrogen or a C₁₋₄ alkyl group, more preferably R²³ is hydrogen);

R²⁴ is selected from the group consisting of hydrogen, a C₁₋₆ alkyl group (the alkyl group optionally being substituted with at least one halogen, —OH, C₁₋₆ alkoxy group, —NH₂, C₁₋₆ alkylamino group or C₁₋₆ dialkylamino group), and —C(O)R²⁵ (preferably R²⁴ is hydrogen or a C₁₋₄ alkyl group, more preferably R²⁴ is hydrogen);

R²⁵ is a C₁₋₆ alkyl group (the alkyl group optionally being substituted with at least one halogen, —OH, C₁₋₆ alkoxy group, —NH₂, C₁₋₆ alkylamino group or C₁₋₆ dialkylamino group) (preferably R²⁵ is a C₁₋₄ alkyl group optionally being substituted with one or two —OH);

R²⁶ is hydrogen or a C₁₋₄ alkyl group;

R²⁷ is selected from the group consisting of halogen, —CN, —NO₂, —NH₂, a C₁₋₆ alkylamino group, a C₁₋₆ dialkylamino group, —OR²⁸, —C(O)OR²⁸, —OC(O)R²⁸, —COR²⁸, —CONR²⁸₂, a C₅₋₇ aryl group (the aryl group optionally being substituted with at least one substituent R²⁹), and a saturated or unsaturated C₅₋₇ heterocyclic group containing at least one heteroatom selected from O, S, and N (R²⁷ is preferably selected from the group consisting of halogen, —CN, —NO₂, —NH₂, a C₁₋₆ alkylamino group, a C₁₋₆ dialkylamino group, —OR²⁸, —C(O)OR²⁸, —OC(O)R¹, —COR²⁸, —CONR²⁸₂; more preferably R²⁷ is halogen);

R²⁸ is hydrogen or a C₁₋₆ alkyl group;

R²⁹ is selected from the group consisting of halogen, —OH, —NH₂, a C₁₋₆ alkylamino group, a C₁₋₆ dialkylamino group, a C₁₋₆ alkoxy group, a C₁₋₆ acyloxy group and a benzyloxy group (R²⁹ is preferably selected from the group consisting of halogen, —OH, —NH₂, a C₁₋₆ alkyl group, a C₁₋₆ alkylamino group, a C₁₋₆ dialkylamino group, and a C₁₋₆ alkoxy group; more preferably R²⁹ is halogen);

n is in the range of 0 to 4 (preferably n is 0, 1 or 2; more preferably the group Y² if present, is meta to the oxazolidinone ring; in a preferred embodiment n is 1);

s is 0, 1 or 2 (preferably s is 0 or 1, more preferably s is 1); and

t is 1 or 2.

In one preferred embodiment of the oxazolidinones of general formula (IIIa) or (IIIb), Q is a C₁₋₆ alkyl group (the alkyl group optionally being substituted with at least one halogen, —CN, —NO₂, —NH₂, C₁₋₆ alkylamino group, C₁₋₆ dialkylamino group or —OR²⁸). More preferably in this embodiment Q is a C₁₋₄ alkyl group. The group Q can be at any position of the phenyl ring, however, it is preferred that Q is para to the oxazolidinone group.

In this embodiment R²² can preferably be selected from the group consisting of hydrogen, a C₁₋₆ alkyl group (the alkyl group optionally being substituted with at least one —OH), a C₅₋₆ cycloalkyl group, a C₅₋₆ cycloalkenyl group, a C₅₋₇ aryl group, and a saturated or unsaturated C₅₋₇ heterocyclic group containing at least one heteroatom selected from O, S and N.

In a second preferred embodiment of general formula (IIIa) or (IIIb) Q is

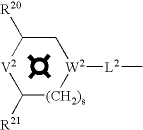

wherein V², W², L², R²⁰, R²¹ and s are as defined above. If V² is —NR²⁴— then can be —C(O)—CH(OH)—CH₂(OH).

Illustrative compounds of general formula (IIIa) are

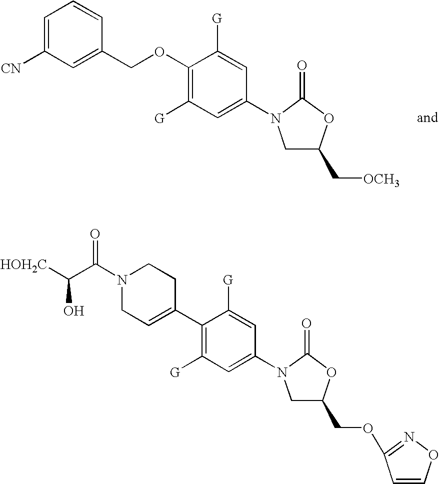

wherein G are independently selected from the group consisting of —H, —F, —Cl, —CH₃ and —CF₃. A further illustrative example is

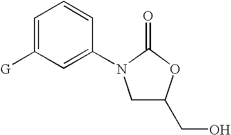

wherein G is selected from the group consisting of —F, —Cl, —CH₃ and —CF₃. This compound can be in the R-configuration at the C5 of the oxazolidinone ring or can be present as a racemate.

In addition to the compounds of general formulae (I), (IIa), (IIb), (IIIa) and (IIIb) themselves, a pharmaceutically acceptable salt thereof is also suitable for use in the present invention. In cases where the compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts. Other salts are well-known to those skilled in the art and may be selected e.g. depending on the desired end use or application form.

The oxazolidinones employed in the present invention can be chiral or achiral compounds. If the oxazolidinones have a chiral center, they can be used in the present invention either as optically pure enantiomers or as racemic mixtures. Preferably the oxazolidinone of general formulae (I), (IIa), (IIb), (IIIa) or (IIIb) is present as an optically pure enantiomer having the S-configuration at C5 of the oxazolidinone ring.

In the oxazolidinones of general formula (I) the group -L-X is preferably present in a position para to the oxazolidinone ring. Corresponding para-configurations are also preferred in the oxazolidinones of the general formulae (IIa), (IIb), (IIIa) and (IIIb).

If present, the groups Y (and the corresponding groups Y¹ and Y²) are preferably in a meta-position on the phenyl ring relative to the oxazolidinone ring.

In the context of the invention the expression “vitamin B2” (which is also commonly known as “riboflavin”) covers all of its pharmaceutically acceptable forms. This includes the actual vitamin itself, any vitamers and pharmaceutically acceptable derivatives. Preferably vitamin B2 is administered as such (i.e. as riboflavin) or in the form of its derivative riboflavin-5′-phosphate.

The term “vitamin B6” means vitamin B6 in all of its pharmaceutically acceptable forms. The term is intended to cover not only its vitamers pyridoxine, pyridoxal and pyridoxamine, but also their pharmaceutically acceptable derivatives such as the respective 5′-phosphates and hydrochlorides. Preferably the vitamin B6 is administered as pyridoxine hydrochloride, pyridoxine, pyridoxal, pyridoxal-5′-phosphate or pyridoxamine; most preferably as pyridoxine hydrochloride or pyridoxal-5′-phosphate.

Similarly the term “vitamin B12” means vitamin B12 in all of its pharmaceutically acceptable forms. It covers all of the closely related cobalamin compounds, which are generically referred to as vitamin B12. In particular, cyanocobalamin and hydroxocobalamin as well as the coenzyme forms adenosylcobalamin and methylcobalamin are herein referred to by the term “vitamin B12”. All vitamers and pharmaceutically acceptable derivatives are covered by this term. Preferably the vitamin B12 is administered as cyanocobalamin, hydroxocobalamin or adenosylcobalamin, more preferably as cyanocobalamin or hydroxocobalamin.

“Folic acid” means folic acid in all of its pharmaceutically acceptable forms. This covers folic acid itself as well as its vitamers and pharmaceutically acceptable derivatives. In particular, folic acid, folates and folinic acid are considered to be covered by the generic term “folic acid”. Preferably the folic acid is administered as folic acid, folates or folinic acid.

“Niacin” (sometimes also commonly known as vitamin B3) means niacin in all of its pharmaceutically acceptable forms. This includes all of the vitamers as well as all pharmaceutically acceptable derivatives. In particular, niacin can be administered in the form of nicotinic acid, nicotinamide and optionally also in the provitamin form of tryptophan, preferably niacin is administered as nicotinic acid or nicotinamide.

A discussion of vitamin B2, vitamin B6, vitamin B12, folic acid and niacin as well as some of their derivatives and vitamers can be found e.g. in Kirk-Othmer, Encyclopedia of Chemical Technology, Vol. 25, 4^{th} edition, John Wiley & Sons, New York, 1998. All of the vitamins mentioned above are commercially available in pharmaceutical grades, e.g. from Hoffmann-La Roche Ltd.

The combination of an oxazolidinone and at least one vitamin is administered to a patient to prevent or treat any oxazolidinone-associated side effects.

Any of the vitamins B2, B6, B12 or folic acid can be administered to the patient either singly or in combination. In one preferred embodiment the patient is given vitamin B2. In another preferred embodiment the patient is given vitamin B6 optionally in combination with niacin. In a further preferred embodiment vitamin B12, folic acid or a combination thereof are administered. It is also possible and preferred to give the patient a mixture of vitamin B2, vitamin B6, vitamin B12, folic acid and optionally niacin. In addition to these vitamins, other vitamins such as vitamin A, vitamin D, vitamin E, vitamin K, vitamin B1, pantothenic acid, biotin and vitamin C can be administered.

The sequence of administering the oxazolidinone and the vitamin(s) depends on the dosage and duration of the oxazolidinone treatment and on whether side effects have already occurred, e.g. caused by oxazolidinone treatment. The best sequence of the oxazolidinone and the vitamin(s) should be determined by the physician based on his medical skill and on the condition of the patient. Generally the vitamin(s) should be administered at least during part of the oxazolidinone treatment. In one embodiment the vitamin(s) can be administered during the whole duration of the oxazolidinone treatment. In another embodiment the vitamin(s) administration can be started when side effects actually occur. The oxazolidinone and the vitamin(s) can be administered concurrently or concomitantly. For example it is possible to first administer the vitamin(s) and then the oxazolidinone or it is also possible to first administer the oxazolidinone and then the vitamin(s). The term “concurrently” means the subject being treated takes one drug within about 15 minutes, preferably within about 5 minutes, of taking the other drug. The term “concomitantly” means the subject being treated takes both drugs within the same treatment period. For example, the duration of the treatment period can be up to 48 hours, preferably up to 24 hours, more preferably up to 12 hours.

If treatment with oxazolidinone is planned, it is possible to give a patient, who might be predisposed to complications, the vitamin(s) several days (e.g. up to 7 days) in advance of the oxazolidinone treatment. Conditions which indicate that the patient might be predisposed to complications are alcohol abuse, cardiac disease, diabetes and renal disease. Furthermore, patients with a multitude of disorders are also liable to complications. The vitamin treatment can then be continued while the oxazolidinone treatment is conducted. It is also possible to administer the vitamin(s) after the oxazolidinone treatment has be concluded. The length of vitamin administration after the oxazolidinone treatment has ended can be determined by a physician depending on the condition of the patient, whether side effects have occurred and the severity of the side effects. The vitamin administration can be continued for as long as the side effects are observable and for some time after they have disappeared (e.g. up to I week or up to 1 month or up to 3 months after the side effects have disappeared). Even if no side effects have been observed, it would be possible to prophylactically continue vitamin administration e.g. for up to one month or for up to one week after the oxazolidinone treatment has ended).

If the oxazolidinone and the vitamin(s) are to be administered concurrently it is possible to include them into the same pharmaceutical composition. However, it is often preferable to administer the oxazolidinone and the vitamin(s) separately so that the dosage and route of application of each component can be individually adapted.

The dosage of the oxazolidinone depends on the condition to be treated. Typical dosages are well-known in the art and the exact dosage can be determined by a physician depending on the severity of the case, the patient's response to the medication and on the fact whether other medications are also being given to the patient.

The desired dose may conveniently be presented in a single dose or be divided into multiple doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye. Also, it is to be understood that the initial dosage administered may be increased beyond the above upper level in order to rapidly achieve the desired plasma concentration. On the other hand, the initial dosage may be smaller than the optimum and the daily dosage may be progressively increased during the course of treatment depending on the particular situation.

In the present invention the oxazolidinone and the vitamin(s) as the active ingredients (either each separately or some/all of the components together) can be administered by any known route of administration. These routes of administration include oral, parenteral, topical, rectal or intranasal administration.

Parenteral administrations include injections to generate a systemic effect or injections directly to the afflicted area. Examples of parenteral administrations are subcutaneous, intravenous, intramuscular, intradermal, intrathecal, intraocular, intravetricular, and general infusion techniques.

Topical administrations include the treatment of infectious areas or organs readily accessibly by local application, such as, for example, eyes, ears including external and middle ear infections, vaginal, open and sutured or closed wounds and skin. Examples of topical systems are patches, gels and creams. It also includes transdermal delivery to generate a systemic effect.

The rectal administration includes the form of suppositories.

The intranasal administration includes nasal aerosol or inhalation applications.

The pharmaceutical compositions may be prepared by methods well known in the art, e.g., by means of conventional mixing, dissolving, granulation, dragee-making, levigating, emulsifying, encapsulating, entrapping, lyophilizing processes or spray drying.

Pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For oral administration, the compounds can be formulated by combining the active compounds with pharmaceutically acceptable carriers well known in the art. Such carriers enable the active ingredient to be formulated as tablets, pills, lozenges, dragees, capsules, liquids, solutions, emulsions, gels, syrups, slurries, suspensions and the like, for oral ingestion by a patient. A carrier can be at least one substance which may also function as a diluent, flavoring agent, solubilizer, lubricant, suspending agent, binder, tablet disintegrating agent, and encapsulating agent. Examples of such carriers or excipients include, but are not limited to, magnesium carbonate, magnesium stearate, talc, sugar, lactose, sucrose, pectin, dextrin, mannitol, sorbitol, starches, gelatin, cellulosic materials, low melting wax, cocoa butter or powder, polymers such as polyethylene glycols and other pharmaceutically acceptable materials.

Dragee cores are provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin, as well as soft, sealed capsules made of gelatin and a plasticizer, such as glycerol or sorbitol. The push-fit capsules can contain the active ingredient in admixture with a filler such as lactose, a binder such as starch, and/or a lubricant such as talc or magnesium stearate and, optionally, stabilizers. In soft capsules, the active ingredient may be dissolved or suspended in a suitable liquid, such as fatty oils, liquid paraffin, liquid polyethylene glycols, cremophor, capmul, medium or long chain mono-, di- or triglycerides. Stabilizers may be added in these formulations, also.

Liquid form compositions include solutions, suspensions and emulsions. For example, there may be provided solutions of the active ingredient dissolved in water and water-propylene glycol and water-polyethylene glycol systems, optionally containing suitable conventional coloring agents, flavoring agents, stabilizers and thickening agents.

The active ingredient may also be formulated for parenteral administration, e.g., by injections, bolus injection or continuous infusion. Formulations for parenteral administration may be presented in unit dosage form, e.g., in ampoules or in multi-dose containers, with an added preservative. The compositions may take such forms as suspensions, solutions or emulsions in oily or aqueous vehicles, and may contain formulating materials such as suspending, stabilizing and/or dispersing agents.

For injection, the active ingredient may be formulated in aqueous solution, preferably in physiologically compatible buffers or physiological saline buffer. Suitable buffering agents include trisodium orthophosphate, sodium bicarbonate, sodium citrate, N-methylglucamine, L(+)-lysine and L(+)-arginine.

The active ingredient or compositions can also be administered e.g. intravenously or intraperitoneally by infusion or injection. Solutions of the active ingredient or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

Pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions can be prepared by incorporating the active ingredient in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Other parenteral administrations also include aqueous solutions of a water soluble form, such as, without limitation, a salt, of the active ingredient. Additionally, suspensions of the active ingredient may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances which increase the viscosity of the suspension, such as sodium carboxymethyl cellulose, sorbitol, or dextran. Optionally, the suspension may also contain suitable stabilizers and/or agents that increase the solubility of the active ingredient to allow for the, preparation of highly concentrated solutions.

Alternatively, the active ingredient may be in a powder form for constitution with a suitable vehicle, e.g., sterile, pyrogen-free water, before use.

For suppository administration, the active ingredient may also be formulated by mixing the agent with a suitable non-irritating excipient which is solid at room temperature but liquid at rectal temperature and therefore will melt in the rectum to release the drug. Such materials include cocoa butter, beeswax and other glycerides.

For administration by inhalation, active ingredient can be conveniently delivered through an aerosol spray in the form of solution, dry powder, or cream. The aerosol may use a pressurized pack or a nebulizer and a suitable propellant. In the case of a pressurized aerosol, the dosage unit may be controlled by providing a valve to deliver a metered amount. Capsules and cartridges of, for example, gelatin for use in an inhaler may be formulated containing a power base such as lactose or starch.

For topical applications, the pharmaceutical composition may be formulated in a suitable ointment containing the active ingredient suspended or dissolved in one or more carriers. Carriers for topical administration of the active ingredient include, but are not limited to, mineral oil, liquid petrolatum, white petrolatum, propylene glycol, polyoxyethylene, polyoxypropylene, emulsifying wax and water. Alternatively, the pharmaceutical compositions can be formulated in a suitable lotion such as suspension, gel, emulsion, or cream containing the active ingredient suspended or dissolved in one or more pharmaceutically acceptable carriers. Suitable carriers include, but are not limited to, mineral oil, sorbitan monostearate, polysorbate 60, cetyl esters wax, ceteary alcohol, 2-octyldodecanol, benzyl alcohol and water. Transdermal delivery devices such as patches are also suitable. In some embodiments the transdermal delivery device may have sustained release characteristics.

For ophthalmic and otitis uses, the pharmaceutical compositions may be formulated as micronized suspensions in isotonic, pH adjusted sterile saline, or preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

In addition to the formulations described previously, the active ingredient may also be formulated as depot preparations. Such long acting formulations may be in the form of implants. The active ingredient may be formulated for this route of administration with suitable polymers, hydrophobic materials, or as a sparingly soluble derivative such as, without limitation, a sparingly soluble salt.

Additionally, the active ingredient may be delivered using a sustained-release system. Various sustained-release materials have been established and are well known by those skilled in the art. Sustained-release capsules may, depending on their chemical nature, release the compounds for 24 hours up to several days. Depending on the chemical nature and the biological stability of the therapeutic reagent, additional strategies for protein stabilization may be employed.

The quantity of the oxazolidinone in the pharmaceutical composition and unit dosage form thereof may be varied or adjusted widely depending upon the particular application, the potency of the particular compound, and the desired concentration. Generally, the quantity of active component will range between 0.5% to 90% by weight of the pharmaceutical composition.

In therapeutic use for treating or combating bacterial infections, the oxazolidinones or pharmaceutical compositions thereof will be administered at a dosage to obtain and maintain a concentration, that is, an amount or blood-level of active oxazolidinone component which will be antibacterially effective. Generally, such antibacterially effective amount of dosage of active oxazolidinone component will be in the range of about 0.1 to about 100 mg/kg of body weight/day, more preferably about 3.0 to about 50 mg/kg of body weight/day. It is to be understood that the dosages may vary depending upon the requirements of the patient, the severity of the bacterial infection being treated, and the particular compound being used. Also, it is to be understood that the initial dosage administered may be increased beyond the above upper level in order to rapidly achieve the desired blood-level or the initial dosage may be smaller than the optimum and the daily dosage may be progressively increased during the course of treatment depending on the particular situation. If desired, the daily dose may also be divided into multiple doses for administration, e.g., 2 to 4 four times per day. Preferably, the oxazolidinone will be administered orally or parenterally, i.e., by injection, for example, by intravenous (i.v.) injection.

If the oxazolidinone is employed as an antidepressant the same general principles mentioned above apply correspondingly. Typically, however, the dosage of oxazolidinones as an antidepressant should be in the range of about 0.1 to about 20 mg/kg of body weight/day, preferably about 0.1 to about 1 mg/kg of body weight/day.

The amount of vitamin(s) administered depends on the nature of the vitamin and also on whether the vitamin(s) is (are) prophylactically administered or if the side effects have already occurred. The exact dosage of the vitamin(s) should be determined by the physician based on the patient's condition and response to the treatment.

Typically the dosage of vitamin B2 can be in the range of about 5 to about 10 mg/day, preferably about 3 to about 5 mg/day.

Vitamin B6 will generally be administered in an amount of about 1 to about 250 mg/day. Therapeutic doses when the side effects are already present will be approximately about 40 to about 200 mg/day, preferably about 50 to about 100, while prophylactic doses will be about 5 to about 20 mg/day.

Usual doses of vitamin B12 will be in the range of about 200 to about 2000 μg/day, preferably about 500 to 1000 μg/day.

Folic acid can be administered in an amount of about 1 to about 10 mg/day, preferably about 1 to about 5 mg/day.

Typically the patients will receive about 20 to about 100 mg/day, preferably about 20 to about 40 mg/day, of niacin.

As discussed above, all of the vitamins can be administered to the patient by any suitable route, preferably they will be in conventional oral or parenteral dosage forms. Such dosage forms are commercially available. Often for prophylaxis the oral dosage form will be preferred, while especially vitamin B6 and vitamin B12 and sometimes vitamin B2 are advantageously administered parenterally (e.g. i.v. or i.m.) in therapeutic doses.

With the present invention, oxazolidinone treatment can now be significantly improved. This is especially advantageous in the case of patients, who require long-term treatment with oxazolidinones or who receive high dosages of these compounds. The cotherapy of oxazolidinones and vitamins is also particularly useful for treating patients who might be predisposed to complications.

What is claimed is:

1. A method of treating a patient in need of oxazolidinone by administering an effective amount of oxazolidinone and an effective amount of at least one vitamin selected from the group consisting of vitamin B2, vitamin B6, vitamin B12 and folic acid.

2. The method of claim 1, wherein the oxazolidinone has the general formula I: 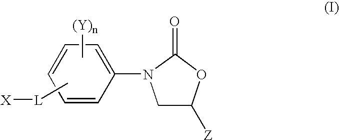
and pharmaceutically acceptable salts thereof;
wherein
X is selected from the group consisting of a C₁₋₁₀ alkyl group (the alkyl group optionally being substituted with at least one substituent R⁴), a C₂₋₁₀ alkenyl group (the alkenyl group optionally being substituted with at least one substituent R⁴), a C₂₋₁₀ alkynyl group (the alkynyl group optionally being substituted with at least one substituent R⁴), a C₃₋₇ cycloalkyl group (the cycloalkyl group optionally being substituted with at least one substituent R⁵), a C₃₋₇ cycloalkenyl group (the cycloalkenyl group optionally being substituted with at least one substituent R⁵), a C₅₋₇ aryl group (the aryl group optionally being substituted with at least one substituent R⁵), and a saturated or unsaturated C₃₋₇ heterocyclic group containing at least one heteroatom selected from O, S, and N (the heterocyclic group optionally being substituted with at least one substituent R⁵);
L is an organic linking group selected from the group consisting of a covalent bond, —O—, —S—, —C(O)—, —C(O)O—, —OC(O)—, —NR¹—, —C(O)NR¹—, —NR¹C(O)—, a C₁₋₄ alkylene group, a C₂₋₄ alkenylene group and a C₂₋₄ alkynylene group (wherein one of the (CH₂) moieties in the alkylene group, alkenylene group or alkynylene group can optionally be replaced by —O—, —S—, —C(O)—, —C(O)O—, —OC(O)—, —NR¹—, —C(O)NR¹— or —NR¹C(O)—);
Y is selected from the group consisting of halogen, —NR¹R², —CN, —NO₂, —OR¹, —SR¹, —S(O)R¹, —S(O)₂R¹, —OS(O)₂R¹, —S(O)₂NR¹R², —NR¹S(O)₂R², —C(O)OR¹, —OC(O)R¹, —COR¹, —CONR¹R², —NR¹COR², a C₁₋₆ alkyl group (the alkyl group optionally being substituted with at least one substituent R³), a C₁₋₆ ether group, a C₁₋₆ thioether group, a C₂₋₆ alkenyl group (the alkenyl group optionally being substituted with at least one substituent R³), and a C₂₋₆ alkynyl group (the alkynyl group optionally being substituted with at least one substituent R³);
n is in the range of 0 to 4;
Z is selected from the group consisting of —CH₂—O—R⁸, —CH₂—NH—C(O)—R⁹, —CH₂—S—R⁸ and —CH₂—NH—C(S)—R⁹; and
R¹, and R² are independently hydrogen or a C₁₋₆ alkyl group (the alkyl group optionally being substituted with at least one halogen, —OH, C₁₋₆ alkoxy group, —NH₂, C₁₋₆ alkylamino group or C₁₋₆ dialkylamino group);
R³ is selected from the group consisting of halogen, —OH, —NH₂, a C₁₋₆ alkylamino group, a C₁₋₆ dialkylamino group, a C₁₋₆ alkoxy group, a C₁₋₆ acyloxy group and a benzyloxy group (R³ is preferably selected from the group consisting of halogen, —OH, —NH₂, a C₁₋₆ alkyl group, a C₁₋₆ alkylamino group, a C₁₋₆ dialkylamino group, and a C₁₋₆ alkoxy group; more preferably R³ is selected from the group consisting of halogen, —OH or a C₁₋₃ alkoxy group; even more preferably R³ is halogen);
R^{3a} is selected from the group consisting of halogen, —OH, —NH₂, a C₁₋₆ alkylamino group, a C₁₋₆ dialkylamino group, a C₁₋₆ alkoxy group, a C₁₋₆ acyloxy group, a benzyloxy group, a C₃₋₆ cycloalkyl group (the cycloalkyl group optionally being substituted with at least one halogen, —OH or —NH₂), a C₃₋₆ cycloalkenyl group (the cycloalkenyl group optionally being substituted with at least one halogen, —OH or —NH₂), a C₅₋₇ aryl group (the aryl group optionally being substituted with at least one halogen, —OH or —NH₂), and a saturated or unsaturated C₃₋₇ heterocyclic group containing at least one heteroatom selected from O, S, and N (the heterocyclic group optionally being substituted with at least one halogen, —OH or —NH₂);
R^{3b} is selected from the group consisting of halogen, —OH, —NH₂, a C₁₋₆ alkylamino group, a C₁₋₆ dialkylamino group, a C₁₋₆ alkoxy group, a C₁₋₆ acyloxy group, a benzyloxy group a C₁₋₆ alkyl group (the alkyl group optionally being substituted with at least one halogen, —OH or —NH₂), a C₁₋₆ ether group, a C₁₋₆ thioether group, a C₂₋₆ alkenyl group (the alkenyl group optionally being substituted with at least one halogen, —OH or —NH₂), and a C₂₋₆ alkynyl group (the alkynyl group optionally being substituted with at least one halogen, —OH or —NH₂);
R⁴ is selected from the group consisting of halogen, —NR¹R², —CN, —NO₂, —OR¹, —SR¹, —S(O)R¹, —S(O)₂R¹, —OS(O)₂R¹, —S(O)₂NR¹R², —NR¹S(O)₂R², —C(O)OR¹, —OC(O)R¹, —COR¹, —CONR¹R², —NR¹COR², a p-toluenesulfonyl group, a C₃₋₆ cycloalkyl group (the cycloalkyl group optionally being substituted with at least one substituent R³), a C₃₋₆ cycloalkenyl group (the cycloalkenyl group optionally being substituted with at least one substituent R³), a C₅₋₇ aryl group (the aryl group optionally being substituted with at least one substituent R³), and a saturated or unsaturated C₃₋₇ heterocyclic group containing at least one heteroatom selected from O, S, and N (the heterocyclic group optionally being substituted with at least one substituent R³);
R⁵ is selected from the group consisting of halogen, —NR¹R², —CN, ═O, ═S, ═N—R¹, —NO₂, —OR¹, —SR¹, —S(O)R¹, —S(O)₂R¹, —OS(O)₂R¹, —S(O)₂NR¹R², —NR¹S(O)₂R², —C(O)OR¹, —OC(O)R¹, —COR¹, —CONR¹R², —NR¹COR², a p-toluenesulfonyl group, a C₁₋₆ alkyl group (the alkyl group optionally being substituted with at least one substituent R³), a C₁₋₆ ether group, a C₁₋₆ thioether group, a C₂₋₆ alkenyl group (the alkenyl group optionally being substituted with at least one substituent R³), a C₂₋₆ alkynyl group (the alkynyl group optionally being substituted with at least one substituent R³), a C₃₋₆ cycloalkyl group (the cycloalkyl group optionally being substituted with at least one substituent R³), a C₃₋₆ cycloalkenyl group (the cycloalkenyl group optionally being substituted with at least one substituent R³), a C₅₋₇ aryl group (the aryl group optionally being substituted with at least one substituent R³), and a saturated or unsaturated C₃₋₇ heterocyclic group containing at least one heteroatom selected from O, S, and N (the heterocyclic group optionally being substituted with at least one substituent R³);
R⁸ is selected from the group consisting of hydrogen, a C₁₋₆ alkyl group (the-alkyl group optionally being substituted with at least one substituent R^{3a}), a C₁₋₆ ether group, a C₁₋₆ thioether group, a C₂₋₆ alkenyl group (the alkenyl group optionally being substituted with at least one substituent R^{3a}), a C₂₋₆ alkynyl group (the alkynyl group optionally being substituted with at least one substituent R^{3a}), a C₃₋₆ cycloalkyl group (the cycloalkyl group optionally being substituted with at least one substituent R^{3b}), a C₃₋₆ cycloalkenyl group (the cycloalkenyl group optionally being substituted with at least one substituent R^{3b}), a C₅₋₇ aryl group (the aryl group optionally being substituted with at least one substituent R^{3b}), and a saturated or unsaturated C₃₋₇ heterocyclic group containing at least one heteroatom selected from O, S, and N (the heterocyclic group optionally being substituted with at least one substituent R^{3b}); and
R⁹ is selected from the group consisting of hydrogen, —NH₂, a C₁₋₆ alkylamino group, a C₁₋₆ dialkylamino group, a C₁₋₆ alkoxy group, a C₁₋₆ alkyl group (the alkyl group optionally being substituted with at least one substituent R^{3a}), a C₁₋₆ ether group, a C₁₋₆ thioether group, a C₂₋₆ alkenyl group (the alkenyl group optionally being substituted with at least one substituent R^{3a}), a C₂₋₆ alkynyl group (the alkynyl group optionally being substituted with at least one substituent R^{3a}), a C₃₋₆ cycloalkyl group (the cycloalkyl group optionally being substituted with at least one substituent R^{3b}), a C₃₋₆ cycloalkenyl group (the cycloalkenyl group optionally being substituted with at least one substituent R^{3b}), a C₅₋₇ aryl group (the aryl group optionally being substituted with at least one substituent R^{3a}), and a saturated or unsaturated C₃₋₇ heterocyclic group containing at least one heteroatom selected from O, S, and N (the heterocyclic group optionally being substituted with at least one substituent R^{3b}).

3. The method of claim 1, wherein the oxazolidinone has the general formula (IIa) or formula (IIb): 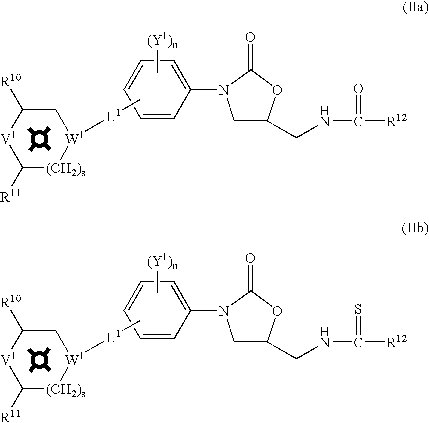
and pharmaceutically acceptable salts thereof,
wherein:
L¹ is selected from the group consisting of a covalent bond, —(CH₂)ₜ— and —(CH₂)ₜ—O—;
V¹ is selected from the group consisting of —O—, —S—, —S(O)—, —S(O)₂—, —C(O)—, —C(S)—, —C(NR¹³)—, —C(H)(NR¹³₂)—, —S(NR¹³)—, —S(O)(NR¹³)—, —C(H)(R¹⁴)—, —C(R¹⁴)═, —NR¹⁴— and —N═;
W¹ is C, CH or N; [symbol]
means that the cyclic group containing V¹ and W¹ can be saturated or unsaturated;
Y¹ is selected from the group consisting of halogen, a C₁₋₄ alkyl group (the alkyl group optionally being substituted with at least one halogen), and a C₁₋₄ alkoxy group;
R¹⁰ is selected from the group consisting of —H, a C₁₋₄ alkyl group, —CN, and —C(O)OR¹⁶;
R¹¹ is —H or a C₁₋₄ alkyl group;
R¹² is selected from the group consisting of hydrogen, a C₁₋₆ alkyl group (the alkyl group optionally being substituted by at least one of the following: halogen, —OH, a C₁₋₆ alkoxy group, a C₁₋₆ acyloxy group, a benzyloxy group, a C₅₋₇ aryl group (the aryl group optionally being substituted with at least one halogen, —OH or —NH₂), and a saturated or unsaturated C₃₋₇ heterocyclic group containing at least one heteroatom selected from O, S, and N (the heterocyclic group optionally being substituted with at least one halogen, —OH or —NH₂)), a C₂₋₆ alkenyl group (the alkenyl group optionally being substituted with at least one of the following: halogen, —OH, a C₁₋₆ alkoxy group, a C₁₋₆ acyloxy group, a benzyloxy group, a C₅₋₇ aryl group (the aryl group optionally being substituted with at least one halogen, —OH or —NH₂), and a saturated or unsaturated C₃₋₇ heterocyclic group containing at least one heteroatom selected from O, S, and N (the heterocyclic group optionally being substituted with at least one halogen, —OH or —NH₂)), —NH₂, a C₁₋₆ alkylamino group, a C₁₋₆ dialkylamino group, a C₁₋₆ alkoxy group, a C₃₋₆ cycloalkyl group, a C₃₋₆ cycloalkenyl group, a C₅₋₇ aryl group, and a saturated or unsaturated C₅₋₇ heterocyclic group containing at least one heteroatom selected from O, S and N;
R¹³ is hydrogen or a C₁₋₆ alkyl group (the alkyl group optionally being substituted with at least one halogen, —OH, C₁₋₆ alkoxy group, —NH₂, C₁₋₆ alkylamino group or C₁₋₆ dialkylamino group);
R¹⁴ is selected from the group consisting of hydrogen, a C₁₋₆ alkyl group (the alkyl group optionally being substituted with at least one halogen, —OH, C₁₋₆ alkoxy group, —NH₂, C₁₋₆ alkylamino group or C₁₋₆ dialkylamino group), and —C(O)R¹⁵;
R¹⁵ is a C₁₋₆ alkyl group (the alkyl group optionally being substituted with at least one halogen, —OH, C₁₋₆ alkoxy group, —NH₂, C₁₋₆ alkylamino group or C₁₋₆ dialkylamino group);
R¹⁶ is hydrogen or a C₁₋₄ alkyl group;
n is in the range of 0 to 4;
s is 0, 1 or 2; and
t is 1 or 2.

4. The method of claim 3 wherein n is 2 and the two groups Y¹ present are meta to the oxazolidinone group and each group Y¹ being independently selected from the group consisting of —F, —Cl, —CH₃ and —CF₃.

5. The method of claim 3 wherein Y¹ is —F.

6. The method of claim 3 wherein n is I and the group Y¹ present is meta to the oxazolidinone group and is —F, —Cl, —CH₃ or —CF₃.

7. The method of claim 3 wherein Y¹ is —F.

8. The method of claim 3 wherein n is 0.

9. The method of claim 3 wherein s is 1.

10. The method of claim 3 wherein V¹ is —O— or —SO₂—.

11. The method of claim 3 wherein W¹ is N.

12. The method of claim 3 wherein L¹ is a covalent bond.

13. The method of claim 3 wherein the ring containing V¹ and W¹ is saturated.

14. The method of claim 3 wherein the oxazolidinone having the general formula (IIa) or (IIb) is an optically pure enantiomer having the S-configuration at C5 of the oxazolidinone ring.

15. The method of claim 1, wherein the oxazolidinone has the general formula (IIIa) or (IIIb): 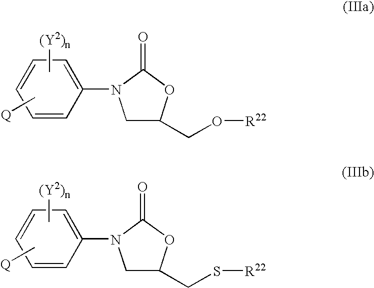
and pharmaceutically acceptable salts thereof,
wherein:
Q is selected from the group consisting of a C₁₋₆ alkyl group (the alkyl group optionally being substituted with at least one substituent R²⁷), a C₂₋₆ alkenyl group (the alkenyl group optionally being substituted with at least one substituent R²⁷), a C₂₋₆ alkynyl group (the alkynyl group optionally being substituted with at least one substituent R²⁷) and 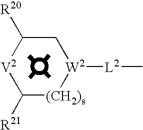
wherein
L² is selected from the group consisting of a covalent bond, —(CH₂)ₜ— and —(CH₂)ₜ—O—;
V² is selected from the group consisting of —O—, —S—, —S(O)—, —S(O)₂—, —C(O)—, —C(S)—, —C(NR²³)_, —C(H)(NR⁰³₂)—, —(NR²³)—, —S(O)(NR²³)—, —C(H)(R²⁴)—, —C(R²⁴)═, —NR²⁴— and —N═;
W² is C, CH or N; [symbol]
means that the cyclic group containing V² and W² can be saturated or unsaturated;
Y² is selected from the group consisting of halogen, a C₁₋₄ alkyl group (the alkyl group optionally being substituted with at least one halogen), and a C₁₋₄ alkoxy group;
R²⁰ is selected from the group consisting of —H, a C₁₋₄ alkyl group, —CN, and —C(O)OR²⁶;
R²¹ is —H or a C₁₋₄ alkyl group;
R²² is selected from the group consisting of hydrogen, a C₁₋₆ alkyl group (the alkyl group optionally being substituted at least one of the following: halogen, —OH, a C₁₋₆ alkoxy group, a C₁₋₆ acyloxy group, —NH₂, a C₁₋₆ alkylamino group, a C₁₋₆ dialkylamino group, a benzyloxy group), a C₃₋₆ cycloalkyl group, a C₃₋₆ cycloalkenyl group, a C₅₋₇ aryl group, and a saturated or unsaturated C₅₋₇ heterocyclic group containing at least one heteroatom selected from O, S and N;
R²³ is hydrogen or a C₁₋₆ alkyl group (the alkyl group optionally being substituted with at least one halogen, —OH, C₁₋₆ alkoxy group, —NH₂, C₁₋₆ alkylamino group or C₁₋₆ dialkylamino group);
R²⁴ is selected from the group consisting of hydrogen, a C₁₋₆ alkyl group (the alkyl group optionally being substituted with at least one halogen, —OH, C₁₋₆ alkoxy group, —NH₂, C₁₋₆ alkylamino group or C₁₋₆ dialkylamino group), and —C(O)R²⁵;
R²⁵ is a C₁₋₆ alkyl group (the alkyl group optionally being substituted with at least one halogen, —OH, C₁₋₆ alkoxy group, —NH₂, C₁₋₆ alkylamino group or C₁₋₆ dialkylamino group);
R²⁶ is hydrogen or a C₁₋₄ alkyl group;
R²⁷ is selected from the group consisting of halogen, —CN, —NO₂, —NH₂, a C₁₋₆ alkylamino group, a C₁₋₆ dialkylamino group, —OR²⁸, —C(O)OR²⁸, —OC(O)R²⁸, —COR²⁸, —CONR²⁸₂, a C₅₋₇ aryl group (the aryl group optionally being substituted with at least one substituent R²⁹), and a saturated or unsaturated C₅₋₇ heterocyclic group containing at least one heteroatom selected from O, S, and N;
R²⁸ is hydrogen or a C₁₋₆ alkyl group;
R²⁹ is selected from the group consisting of halogen, —OH, —NH₂, a C₁₋₆ alkylamino group, a C₁₋₆ dialkylamino group, a C₁₋₆ alkoxy group, a C₁₋₆ acyloxy group and a benzyloxy group;
n is in the range of 0 to 4;
s is 0, 1 or 2; and
t is 1 or 2.

16. The method of claim 15 wherein R²² is selected from the group consisting of hydrogen, a C₁₋₆ alkyl group (the alkyl optionally being substituted at least one —OH), a C₅₋₆ cycloalkyl group, a C₅₋₆ cycloalkenyl group, a C₅₋₇ aryl group, and a saturated or unsaturated C₅₋₇ heterocyclic group containing at least one heteroatom selected from O, S and N.

17. The method of claim 15 wherein Q is a C₁₋₆ alkyl group (the alkyl group optionally being substituted with at least one substituent selected from the group consisting of halogen, —CN, —NO₂, —NH₂, C₁₋₆ alkylamino group, C₁₋₆ dialkylamino group or —OR²⁸ wherein R²⁸ is as defined in claim 15).

18. The method of claim 15 wherein Q is 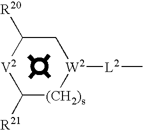
and wherein V¹, W¹, L², R²⁰, R²¹ and s are as defined in claim 15.

19. The method of claim 18 wherein V² is —NR²⁴— and R²⁴ is —C(O)—CH(OH)—CH₂(OH).

20. The method of claim 18 wherein the cyclic group containing V² and W² contains at least one unsaturated bond.

21. The method of claim 18 wherein L² is selected from the group consisting of a covalent bond, —CH₂—CH₂— and —CH₂—O—.

22. The method of claim 18 wherein L² is a covalent bond.

23. The method of claim 15 wherein n is 2 and the two groups Y² present are meta to the oxazolidinone group and each group Y² being independently selected from the group consisting of —F, —Cl, —CH₃ and —CF₃.

24. The method of claim 23 wherein Y² is —F.

25. The method of claim 15 wherein n is 1 and the group Y² present is meta to the oxazolidinone group and is —F, —Cl, —CH₃ or —CF₃.

26. The method of claim 25 wherein Y² is —F.

27. The method of claim 15 wherein n is 0.

28. The method of claim 18 wherein s is 1.

29. The method of claim 15 wherein the oxazolidinone having the general formula (IIIa) or (IIIb) is an optically pure enantiomer having the S-configuration at C5 of the oxazolidinone ring.

30. The method of claim 15 wherein the oxazolidinone is 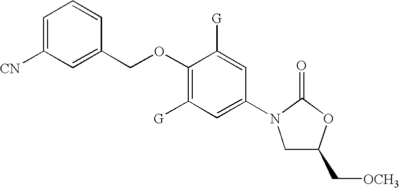
wherein G are independently selected from the group consisting of —H, —F, —Cl, —CH₃ and —CF₃.

31. The method of claim 15 wherein the oxazolidinone is 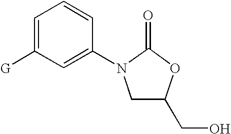
wherein G is selected from the group consisting of —F, —Cl, —CH₃ and —CF₃.

32. The method of claim 31 wherein the oxazolidinone a racemate or an optically pure enantiomer having the R-configuration at C5 of the oxazolidinone ring.

33. The method of claim 15 wherein the oxazolidinone is 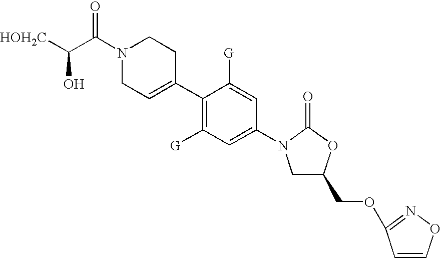
wherein G are independently selected from the group consisting of —H, —F, —Cl, —CH₃ and —CF₃.

34. The method of claim 1 wherein the oxazolidinone is 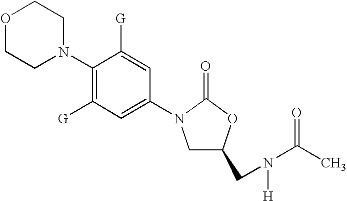
wherein G are independently —H, —F, —Cl, —CH₃ or —CF₃.

35. The method of claim 1, wherein the oxazolidinone is linezolid, furazolidone or toloxatone.

36. The method of claim 35, wherein the oxazolidinone is linezolid.

37. The method of claim 1, wherein the vitamin is vitamin B2.

38. The method of claim 1 wherein the vitamin is vitamin B6.

39. The method of claim 38 further comprising the step of administering niacin.

40. The method of claim 1 wherein the vitamin is vitamin B12.

41. The method of claim 1 wherein the vitamin is folic acid.

42. The method of claim 1 wherein the oxazolidinone and the vitamin are administered concurrently.

43. The method of claim 1 wherein the oxazolidinone and the vitamin are administered concomitantly.

44. The method of claim 1 wherein the vitamin is administered before administering the oxazolidinone.

45. The method of claim 1 wherein the vitamin is administered after administering the oxazolidinone.

46. The method of claim 1 wherein the oxazolidinone is administered in amount of about 0.1 to about 100 mg/kg of body weight/day.

47. The method of claim 1 wherein the vitamin B2 is administered in an amount of about 1 to 10 mg/day.

48. The method of claim 1 wherein the vitamin B6 is administered in an amount of about 1 to about 250 mg/day.

49. The method of claim 1 wherein the vitamin B12 is administered in an amount of about 200 to about 2000 μg/day.

50. The method of claim 1 wherein the folic acid is administered in an amount of about 1 to about 10 mg/day.

51. The method of claim 1 wherein the oxazolidinone is administered orally, parenterally, topically, rectally or intranasally.

52. The method of claim 51 wherein the oxazolidinone is administered by intravenous injection.

53. The method of-claim 1 wherein the vitamin is administered orally, parenterally, topically, rectally or intranasally.

54. A method of treating or preventing an oxazolidinone-associated side effect by administering an effective amount at least one vitamin selected from the group consisting of vitamin B2, vitamin B6, vitamin B12 and folic acid to a patient in need thereof.

55. A method of treating or preventing oxazolidinone-associated normocytic anemia by administering an effective amount of vitamin B2 to a patient in need thereof.

56. A method of treating or preventing oxazolidinone-associated peripheral sensory neuropathy by administering an effective amount of vitamin B2 to a patient in need thereof.

57. A method of treating or preventing oxazolidinone-associated sideroblastic anemia by administering an effective amount of vitamin B6 to a patient in need thereof.

58. A method of treating or preventing oxazolidinone-associated peripheral sensory neuropathy by administering an effective amount of vitamin B6 to a patient in need thereof.

59. A method of treating or preventing oxazolidinone-associated optic neuropathy by administering an effective amount of vitamin B6 to a patient in need thereof.

60. A method of treating or preventing oxazolidinone-associated seizures by administering an effective amount of vitamin B6 to a patient in need thereof.

61. A method of treating or preventing oxazolidinone-associated thrombocytopenia by administering an effective amount of vitamin B6 to a patient in need thereof.

62. A method of treating or preventing oxazolidinone-associated cheilosis by administering an effective amount of vitamin B6 to a patient in need thereof.

63. A method of treating or preventing oxazolidinone-associated seborrheic dermatitis by administering an effective amount of vitamin B6 to a patient in need thereof.

64. A method of treating or preventing oxazolidinone-associated hyporegenerative anemia by administering an effective amount at least one vitamin selected from the group consisting of vitamin B12 and folic acid to a patient in need thereof.

65. A method of treating or preventing oxazolidinone-associated megaloblastic anemia by administering an effective amount at least one vitamin selected from the group consisting of vitamin B12 and folic acid to a patient in need thereof.

66. A method of treating or preventing a bacterial infection by administering an effective amount of oxazolidinone and an effective amount of at least one vitamin selected from the group consisting of vitamin B2, vitamin B6, vitamin B12 and folic acid.

67. A method of treating or preventing a depressive disorder by administering an effective amount of oxazolidinone and an effective amount of at least one vitamin selected from the group consisting of vitamin B2, vitamin B6, vitamin B12 and folic acid.

68. A pharmaceutical composition comprising (a) oxazolidinone and (b) at least one vitamin selected from the group consisting of vitamin B2, vitamin B6, vitamin B12 and folic acid.

69. A medical kit comprising (a) oxazolidinone and (b) at least one vitamin selected from the group consisting of vitamin B2, vitamin B6, vitamin B12 and folic acid.
